(19) United States
(12) Patent Application Publication
(10) Pub. No.: US 2025/0025093 A1
(43) Pub. Date: Jan. 23, 2025

(54) CONNECTED NEURAL STIMULATOR INTERFACE, CONNECTED-OBJECT MANAGER, CONNECTED ELECTRONIC DEVICE, METHOD FOR STARTING UP A CONNECTED NEURAL STIMULATOR INTERFACE, AND METHOD FOR CONTROLLING AN ELECTRONIC DEVICE

(71) Applicant: ORANGE, Issy-les-Moulineaux (FR)

(72) Inventors: Foued Bouchnak, CHATILLON CEDEX (FR); Cyril Plapous, CHATILLON CEDEX (FR)

(21) Appl. No.: 18/713,891

(22) PCT Filed: Nov. 28, 2022
(86) PCT No.: PCT/EP2022/083445

§ 371 Date: May 28, 2024

(30) Foreign Application Priority Data
Nov. 30, 2021 (FR) FR2112729

(51) Int. Cl. A61B 5/378

(52) U.S. Cl. CPC A61B 5/378

(57) ABSTRACT

A neural stimulator interface connected to a communication network, a connected-object manager, a direct neural interface, an electronic device connected to the communication network, a method for initializing a connected neural stimulator interface, a method for controlling a connected electronic device, and in particular, an interaction with an electronic device via a direct neural interface. A neural stimulator interface connected to a communication network is capable of reproducing a visual signal with a given frequency. The given frequency is associated with a given command of an electronic device connected to the communication network upon initializing the neural stimulator interface. The connected neural stimulator interface is separate from the electronic device. Thus, an electronic device that does not have a display can be granted a so-called BCI function, i.e. a neural interface, by using the connected neural stimulator interface.

### TECHNICAL FIELD

The invention relates to a neuronal stimulator interface connected to a communications network, a connected-object manager, a direct neuronal interface, an electronic device connected to the communications network, a method for initializing a connected neuronal stimulator interface and a method for controlling a connected electronic device. In particular, the invention relates to the interaction with an electronic device by means of a direct neuronal interface.

### PRIOR ART

Direct neuronal interfaces have been introduced in order to allow the accessibility of the greatest number of people, in particular of those suffering from a physical disability, to electronic devices.

Currently, electronic devices must be equipped with a screen for reproducing a specific stimulus. This stimulus triggers the appearance of a particular signal, also called visually evoked potential, within the signals of the electroencephalogram (EEG) when the subject is subjected to the visual stimulation with this stimulus. A specific device, for example a BCI or “Brain Computer Interface” headset, in other words a direct neuronal interface headset allowing the EEG signals to be measured, detects within these EEG signals a frequency which corresponds to the frequency of the visual stimulus.

Virtual and/or augmented reality headsets are furthermore beginning to implement direct neuronal interfaces. Thus, a greater number of users, including those without a disability, may be able to use the direct neuronal interface in order to facilitate, to simplify, the interaction with electronic devices, thus reducing, including for them, the errors in interactions with the devices.

However, direct neuronal interfaces only allow interaction with certain electronic devices, also called BCI-compatible electronic devices, fabricated specifically so as to be connected to a direct neuronal interface, such as a BCI headset, in particular to increase the accessibility of these BCI-compatible electronic devices. Notably, these BCI-compatible electronic devices must be equipped with a screen allowing the reproduction of visual stimuli with a given frequency and with a controller designed to control the electronic device according to the frequency received from the direct neuronal interface.

The non-BCI-compatible electronic devices will not be able to be controlled by means of a neuronal interface rendering their use more complex (thus leading to interaction errors) for all, or even impossible for some people. In particular, the electronic devices designed to be controlled by one or more buttons (irrespective of the type of physical button: push-button, touch-operated, etc.) requires the user to be able to correctly operate these buttons, which may be prevented either by his/her disability or because his/her hands are hindered either by clothing (for example, gloves) or by an object. Owing to this impediment, the device may trigger an erroneous action, either because the user has actuated the wrong button, or the user has not actuated any button following a request from the device.

### DESCRIPTION OF THE INVENTION

One of the aims of the present invention is to overcome the drawbacks of the prior art.

One subject of the invention is a neuronal stimulator interface connected to a communications network designed to reproduce a visual signal with a given frequency fn, the given frequency being associated with a given command of an electronic device connected to the communications network during the initialization of the neuronal stimulator interface, in which the connected neuronal stimulator interface is distinct from the electronic device.

Thus, an electronic device which is not equipped with a screen may be given a function called BCI, in other words a neuronal interface function, by means of the connected neuronal stimulator interface according to the invention.

Advantageously, the given command is identical to a given command triggered by a physical button of the electronic device, the connected neuronal stimulator interface being physically associated with the physical button.

Thus, the BCI interaction errors are reduced since the connected neuronal stimulator interface which reproduces the visual stimulus for the direct neuronal interaction is physically associated with the physical button of the interaction to be triggered. Hence, the user looking at the button with which he/she wishes to interact therefore sees, at the same time, the visual signal reproduced by the physically associated connected neuronal stimulator interface, which triggers an EEG signal with a frequency corresponding to the given frequency of the visual signal reproduced by the connected neuronal stimulator interface, itself associated with the given command of the physical button.

Advantageously, the connected neuronal stimulator interface comprises a fastening designed to keep the connected neuronal stimulator interface in relation to the physical button.

Thus, the connected neuronal stimulator interface cannot move, limiting the interaction errors linked to the loss of the physical association of the connected neuronal stimulator interface with the physical button the given command of which is associated with the given frequency of the visual signal reproduced by the connected neuronal stimulator interface.

Advantageously, the fastening is composed of two separable parts:
a first part referred to as support designed to keep the connected neuronal stimulator interface in relation to the physical button and
a second part fixable onto the support: an interface proper designed to reproduce a visual signal with a given frequency comprising the second part of the fastening.

Thus, the connected neuronal stimulator interface may be moved notably in order to be recharged and repositioned exactly in physical association with the physical button with which it was initially associated.

Advantageously, the connected neuronal stimulator interface comprises a rechargeable battery.

Another subject of the invention is a manager of objects that are connected to a communications network including at least one connected neuronal stimulator interface and one electronic device, in which the connected-object manager comprises a coupler designed to associate a given frequency of a visual signal reproduced by a neuronal stimulator interface connected to the communications network with a given command of an electronic device connected to the communications network.

A further subject of the invention is a manager of objects that are connected to a communications network which has at least one connected neuronal stimulator interface and one electronic device, in which the connected-object manager comprises a controller designed to control an electronic device connected to the communications network as a function of a neuronal frequency received from a direct neuronal interface when a visual signal with a given frequency is reproduced by the connected neuronal stimulator interface, the received neuronal frequency being a function of the given frequency fn.

Advantageously, the connected-object manager comprises the coupler of a given frequency of a visual signal reproduced by the connected neuronal stimulator interface to a given command of an electronic device and the connected electronic device controller as a function of a neuronal frequency received from a direct neuronal interface when the visual signal is reproduced at the given frequency by the connected neuronal stimulator interface.

Advantageously, the connected-object manager comprises a transmitter exchanging a signal relating to the given frequency with the connected neuronal stimulator interface.

Advantageously, the signal relating to the given frequency is a signal from amongst the following signals:
a signal indicating the given frequency sent out by the connected-object manager to the connected neuronal stimulator interface;
a visual signal to be reproduced with the given frequency generated by the connected-object manager and sent by the connected-object manager to the connected neuronal stimulator interface;
a signal indicating the given frequency sent out by the connected neuronal stimulator interface to the connected-object manager.

One subject of the invention is, furthermore, a direct neuronal interface designed to capture a frequency relating to a user during a reproduction of a visual signal with a given frequency, in which the direct neuronal interface comprises an emitter of the captured neuronal frequency via a communications network, the emitter of the captured neuronal frequency being designed to trigger a given command of an electronic device associated, during an initialization of the neuronal stimulator interface, with the given frequency of a visual signal reproduced by a neuronal stimulator interface connected to the communications network, the emitted neuronal frequency being a function of the given frequency.

Another subject of the invention is an electronic device connected to a communications network, in which the electronic device comprises an actuator designed to control a component of the electronic device according to at least one command, the given command being a function of a neuronal frequency emitted by a direct neuronal interface when a visual signal with a given frequency is reproduced by a connected neuronal stimulator interface, the emitted neuronal frequency being a function of the given frequency, the given frequency and the given command being associated during an initialization of the neuronal stimulator interface.

Advantageously, the electronic device comprises a power supply of the connected neuronal stimulator interface when the connected neuronal stimulator interface is associated with a physical button of the electronic device.

Advantageously, the power supply of the connected neuronal stimulator interface is a power supply from amongst the following power supplies:
a power supply connector designed to be connected to the connected neuronal stimulator interface when the connected neuronal stimulator interface is associated with a physical button of the electronic device;
a charger by induction designed to recharge a battery of the connected neuronal stimulator interface when the connected neuronal stimulator interface is associated with a physical button of the electronic device.

Yet another subject of the invention is a method for initializing a neuronal stimulator interface connected to a communications network and an electronic device connected to the communications network, in which the initialization method comprises a pairing associating a given frequency of a visual signal reproduced by a neuronal stimulator interface connected to the communications network with a given command of an electronic device connected to the communications network.

A further subject of the invention is a method for controlling an electronic device, in which the control method controls an electronic device connected to a communications network as a function of a neuronal frequency received from a direct neuronal interface when a visual signal with a given frequency is reproduced by a neuronal stimulator interface connected to the communications network, the received neuronal frequency being a function of the given frequency, the given frequency and the given command being associated during an initialization of the neuronal stimulator interface.

Advantageously, according to one embodiment of the invention, the various steps of the methods according to the invention are implemented by a software application or computer program, this software comprising program instructions intended to be executed by a data processor of a device forming part of a connected-object manager and being designed to control the execution of the various steps of this method.

The invention is also aimed at a program comprising program code instructions for the execution of the steps of the method for initializing a connected neuronal stimulator interface and/or of the method for controlling an electronic device when said program is executed by a processor.

This program may use any given programming language and may take the form of source code, object code or code intermediate between source code and object code such as in a partially compiled form or in any other desirable form.

### BRIEF DESCRIPTION OF THE DRAWINGS

The features and advantages of the invention will become more clearly apparent upon reading the description, presented by way of example, and from the related figures which show:

FIG. 1, a simplified diagram of an architecture comprising a connected neuronal stimulator interface and an electronic device according to the invention,

FIG. 2, a simplified diagram of an architecture comprising a connected-object manager including a connected neuronal stimulator interface and an electronic device according to the invention,

FIG. 3, a simplified diagram of exchanges during the implementation of a method for initializing a connected neuronal stimulator interface according to the invention,

FIG. 4, a simplified diagram of exchanges during the implementation of a method for controlling an electronic device using a connected neuronal stimulator interface according to the invention,

FIG. 5a, a simplified diagram of an electronic device for which the invention may be used,

FIG. 5b, a simplified diagram of a first physical association of connected neuronal stimulator interfaces with an electronic device according to the invention,

FIG. 5c, a simplified diagram of a second physical association of connected neuronal stimulator interfaces with an electronic device according to the invention,

FIG. 6a, a simplified diagram of a first variant of a connected neuronal stimulator interface comprising a fastening by bonding according to the invention,

FIG. 6b, a simplified diagram of a second variant of a connected neuronal stimulator interface comprising a clip-on attachment system according to the invention,

FIG. 6c, a simplified diagram of a third variant of a connected neuronal stimulator interface comprising a first part designed to reproduce a visual signal with a given frequency and a second part comprising a fastening according to the invention.

### DESCRIPTION OF THE EMBODIMENTS

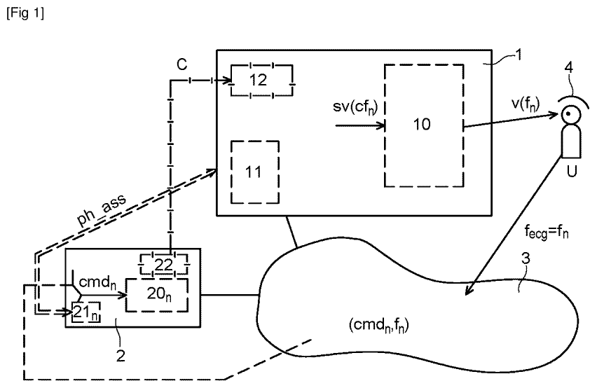

FIG. 1 illustrates a simplified diagram of an architecture comprising a connected neuronal stimulator interface and an electronic device according to the invention.

A neuronal stimulator interface 1 connected to a communications network 3 is designed to reproduce a visual signal sv(fn) with a given frequency fn. The given frequency fn is associated with a given command cmdₙ of an electronic device 2 connected to the communications network 3 during an initialization of the neuronal stimulator interface. The connected neuronal stimulator interface 1 is distinct from the electronic device 2.

Thus, an electronic device 2 which is not equipped with a screen or other types of visual reproduction device may have a function referred to as BCI added, in other words a neuronal interface function, by means of the connected neuronal stimulator interface according to the invention.

In particular, the connected neuronal stimulator interface 1 comprises a visual reproduction device 10 designed to reproduce the visual signal sv(fn) with the given frequency fn. The visual reproduction device 10 is notably a screen, 2D, 3D, etc. (the screen comprising at least one pixel able to vary, notably turn on or off, at at least one predefined frequency), one or more light-emitting or laser diodes (notably a mosaic of light-emitting diodes), a holographic display, etc.

The connected neuronal stimulator interface 1, in particular the reproduction device 10, displays a visual stimulus v(fn) with the given frequency corresponding to the reproduction of the visual signal sv(fn) with the given frequency. The visual stimulus v(fn) can be seen by a user U.

In particular, the connected neuronal stimulator interface 1 and the electronic device 2 are two devices that are distinct in that the connected neuronal stimulator interface 1 and the electronic device 2 are separate. The connected neuronal stimulator interface 1 being distinct from the electronic device 2 is notably understood to mean that the electronic device 2 does not comprise or does not implement the connected neuronal stimulator interface 1.

In particular, the given command cmdₙ is identical to a given command triggered by a physical button 21n of the electronic device 2. The connected neuronal stimulator interface 1 is physically associated ph_ass with the physical button 21n.

Physical association is understood to mean that the connected neuronal stimulator interface is positioned in geographical proximity to the physical button or to what is indicative of the physical button with which it is associated. Geographical proximity is understood to mean that the connected neuronal stimulator interface is placed directly on the physical button, or at a distance from the associated physical button less than the distance with the physical button of the electronic device nearest to the associated physical button, or again, if the electronic device is equipped with several devices placed on the same straight line, on a straight line perpendicular to this straight line going through the associated physical button, etc.

Thus, the BCI interaction errors are reduced since the connected neuronal stimulator interface which reproduces the visual stimulus for the direct neuronal interaction is physically associated with the physical button of the interaction to be triggered. Hence, the user looking at the button with which he/she wishes to interact therefore sees, at the same time, the visual signal sv(fn) reproduced by the physically associated connected neuronal stimulator interface, which triggers an EEG signal with a frequency corresponding to the given frequency fn of the visual signal sv(fn) reproduced by the connected neuronal stimulator interface, itself associated with the given command of the physical button.

A conventional physical button notably operating by physically pressing or by touch detection, the connected neuronal stimulator interface according to the invention adds to the existing button a capacity for emission of visual stimuli, in particular lit up, notably by displaying such a visual stimulus. Since the visual stimulus can be detected in the EEG signals by means of a direct neuronal interface, for example implemented in a BCI headset, the invention thus allows a user to interact with the electronic device as if he/she were interacting with the conventional physical button through an additional mode of interaction.

Thus, everything happens as if the user were actuating the physical button without touching it, or even making physical contact, by only using the EEG signals.

A physical button 21n is defined by opposition to a virtual button. The physical button 21n is notably a push-button, a touch-sensitive button, an optical button, etc. The physical button 21n allows one or more commands to be triggered. In the case of a multi-command physical button, the multi-command button is notably:
a button switching between several contactors. For example, the switch button switches between two positions triggering two separate, potentially opposing, commands: turning on/off a component of the electronic device, or a cruciform button offering four positions triggering four separate commands of the same type, notably commands for different movements: forward/back/right/left movements, etc.;
a rotary button between several positions for separate commands: notably various predefined values of a given command: for example for temperature, for volume, etc. or several modes of operation of the electronic device, and/or of a component of the electronic device, such as different modes of operation of a connected oven: grill, rotary heating, etc. or activation of a component of this connected oven: turning on the light, updating the clock, etc.

A separate connected neuronal stimulator interface will be associated with each position of the multi-command button. A first connected neuronal stimulator interface associated with a first position of the multi-command button will reproduce a visual signal sv(fn) with a first given frequency fn distinct from the second given frequency fn used for reproducing a visual signal sv(fn) by a second connected neuronal stimulator interface associated with a second position of the multi-command button. The first connected neuronal stimulator interface and the second connected neuronal stimulator interface, and respectively the associated first position of the multi-command button and second position of the multi-command button, are distinct from one another.

In particular, the connected neuronal stimulator interface 1 comprises a fastening 11 designed to keep the connected neuronal stimulator interface 1 in relation to the physical button 21n.

Thus, the connected neuronal stimulator interface cannot move, limiting the interaction errors linked to the loss of the physical association of the connected neuronal stimulator interface with the physical button the given command of which is associated with the given frequency fn of the visual signal sv(fn) reproduced by the connected neuronal stimulator interface.

Advantageously, the connected neuronal stimulator interface 1 comprises a rechargeable battery 12. Notably, the connected neuronal stimulator interface 1 may be connected as a power supply to any given electronic device such as the electronic device 2, a computer, a tablet, etc. notably by means of a USB cable in order to be recharged, or even to an electrical socket via a battery charger.

An electronic device 2 connected to a communications network 3 comprises an actuator 20n designed to control a component of the electronic device 2 as a function of at least one given command cmdₙ. The given command cmdₙ depends on a neuronal frequency f_{ecg} emitted by a direct neuronal interface 4 when a visual signal sv(fn) is reproduced with a given frequency fn by a connected neuronal stimulator interface 1. The emitted neuronal frequency f_{ecg} is a function of the given frequency fn, the given frequency and the given command being associated during an initialization of the neuronal stimulator interface.

Notably, the actuator 20n is a processor 20n designed to execute a processing operation according to at least one command.

In particular, the electronic device 2 comprises a power supply of the connected neuronal stimulator interface 22 when the connected neuronal stimulator interface 1 is associated with a physical button 21n of the electronic device 2.

In particular, the power supply 22 implemented in the electronic device 2 recharges c the battery 12 of the connected neuronal stimulator interface 1 notably when the connected neuronal stimulator interface 1 is connected to the electronic device 2, for example by a cable, by induction, etc.

In particular, the power supply of the connected neuronal stimulator interface 22 is a power supply from amongst the following power supplies:
a power supply connector designed to be connected to the connected neuronal stimulator interface when the connected neuronal stimulator interface is associated with a physical button of the electronic device;
a charger by induction designed to recharge a battery of the connected neuronal stimulator interface when the connected neuronal stimulator interface is associated with a physical button of the electronic device.

A direct neuronal interface 4 is designed to capture a frequency f_{ecg} relating to a user U when a visual signal sv(fn) is reproduced with a given frequency fn. The direct neuronal interface 4 comprises an emitter 44 (not shown) of the captured neuronal frequency f_{ecg} via a communications network 3. The emitter of the captured neuronal frequency is designed to trigger a given command cmdₙ of an electronic device 2 associated, during an initialization of the neuronal stimulator interface, with the given frequency fn of a visual signal sv(fn) reproduced by a neuronal stimulator interface 1 connected to the communications network 3. The transmitted neuronal frequency f_{ecg} is a function of the given frequency fn.

In particular, the emitter 44 is a communication interface with the communications network 3.

In particular, the given frequency fn being associated with a given command cmdₙ of the electronic device 2, the captured neuronal frequency f_{ecg} corresponding to the given frequency fn of the reproduced visual signal observed by the user U is therefore associated with this same command cmdₙ of the electronic device 2. The captured neuronal frequency is accordingly able to control cmdₙ the electronic device.

In particular, the emitter 44 is designed to provide the captured neuronal frequency f_{ecg} relating to a user U when a visual signal sv(fn) is reproduced with a given frequency fn by a neuronal stimulator interface 1 connected to a communications network 3.

The captured neuronal frequency f_{ecg} is a frequency of a visually evoked potential signal within signals of the electroencephalogram of a user U.

A given frequency fn of reproduction of a visual signal by a neuronal stimulator interface 1 connected to a communications network 3 is associated with a given command cmdₙ of an electronic device 2 connected to the communications network 3. The connected neuronal stimulator interface 1 and the electronic device are distinct from one another.

Potentially, the connected neuronal stimulator interface 1 is physically associated ph_ass with a physical button 21n of the electronic device, a manipulation of the physical button triggering the given command cmdₙ.

In particular, the reproduction device 10 of the connected neuronal stimulator interface 1 reproduces a visual signal sv(fn) with the given frequency.

When the user U looks at the visual stimulus v(fn) resulting from the reproduction of the visual signal sv(fn) with the given frequency fn by the connected neuronal stimulator interface 1, a direct neuronal interface 4 captures a neuronal frequency f_{ecg} relating to the user U. The captured neuronal frequency f_{ecg} and the given frequency fn have the same value.

In particular, the direct neuronal interface 4 is implemented in a BCI headset or an augmented reality headset which then possesses EEG capacities, in other words capacities for detection of electroencephalogram signals.

The direct neuronal interface 4 supplies the captured neuronal frequency f_{ecg} via the communications network 3. A given command cmdₙ being a function of the captured neuronal frequency is implemented by the electronic device 2.

Potentially, the electronic device 2 receives from an analyzer (not illustrated in FIG. 1), as a function of the neuronal frequency emitted by the direct neuronal interface 4 and of the pair associating a given command and a given frequency (cmdₙ,fₙ), the given command cmdₙ. The analyzer is notably implemented in a device of the communications network 3 distinct from the neuronal stimulator interface 1 and/or from the electronic device 2 and/or from the direct neuronal interface 4.

In one particular embodiment using an augmented reality headset (not shown) implementing the direct neuronal interface 4, the augmented reality headset furthermore comprises at least one connected neuronal stimulator interface 1.

Thus, the augmented reality headset reproduces within a given geographical area associated with the electronic device 2, in particular with a physical button 21n of this electronic device 2, the visual signal sv(fn) with a given frequency associated with the given command cmdₙ of the electronic device 2, which may notably be activated by means of the physical button 21n.

In particular, the reproduction device 10 of the connected neuronal stimulator interface 1 is the reproduction device of the augmented reality headset implemented for the reproduction within the given geographical area.

The augmented reality headset implementing connected neuronal stimulator interfaces thus allows several connected neuronal stimulator interfaces to be easily associated for a given electronic device but also connected neuronal stimulator interfaces to be associated with a large number of electronic devices. This increases the possibilities of equipping electronic devices (connected sockets, connected switches, hi-fi, connected speakers, home router, TV decoder, DVD player, connected home appliances, remote control, keyboard, bus stop request button, etc.) with means of neuronal interactions, but especially the number of electronic devices equipped with means of neuronal interactions in one location, such as a home, an office, etc.

In particular, the connected neuronal stimulator interface 1 is connected to the communications network 3 via a local wireless network such as Wifi, Bluetooth, Lora, etc.

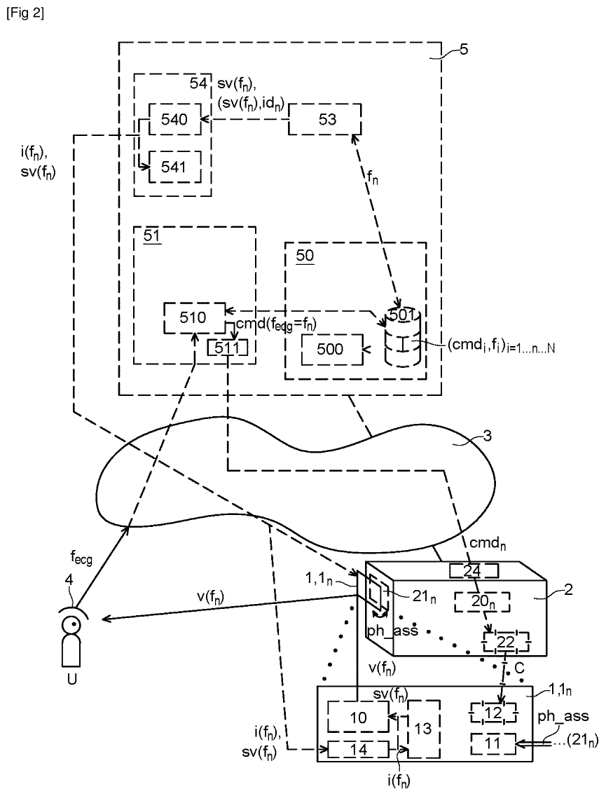

FIG. 2 illustrates a simplified diagram of an architecture comprising a connected-object manager including a connected neuronal stimulator interface and an electronic device according to the invention.

Potentially, a manager 5 of objects connected to a communications network 3 including at least one connected neuronal stimulator interface 1, 1n and one electronic device 2 is used for implementing the invention.

An object is understood to mean any solid thing considered as a whole, fabricated and intended for a certain use. A connected object is then an object capable of exchanging a signal via a home communications network (via a wired LAN network or wireless Wifi, Bluetooth, Lora, etc.) and/or remote communications network such as wired Internet (ADSL, fiber, etc.) or wireless (4G, 5G, etc.) with at least one electronic device such as a service provision device.

A connected object is understood to mean physical or virtual objects interconnected via at least one local and/or remote communications network for providing the latest services by virtue of information and/or communications technologies.

The communications network 3 is a remote or local, home network.

In particular, the connected neuronal stimulator interface 1 is connected to a network referred to as Internet of Things, or IoT, managed by the connected-object manager 5. The IoT network is notably associated with a specific location, such as the home of the user, a workshop and/or a company building, etc.

In particular, a connected-object manager 5 comprises a coupler 50 designed to associate a given frequency fn of a visual signal sv(fn) reproduced by a neuronal stimulator interface 1, 1n connected to the communications network 3 with a given command cmdₙ of an electronic device 2 connected to the communications network 3.

In particular, the coupler 50 comprises a storage device 501 for given command-given frequency pairs (cmdᵢ, fi)_{i=1 . . . . N}. The storage device 501 is a memory, a command database, etc.

In particular, the coupler 50 comprises a recorder 500 designed to store the given command—given frequency pair (cmdₙ,fₙ), notably in a storage device 501 of the connected-object manager 50.

In particular, a connected-object manager 5 comprises a controller 51 designed to control an electronic device 2 connected to the communications network 3 according to a neuronal frequency f_{ecg} received from a direct neuronal interface 4 when a visual signal sv(fn) is reproduced with a given frequency fn by the connected neuronal stimulator interface 1, 1n, the received neuronal frequency f_{ecg} being a function of the given frequency fn.

In particular, the controller 51 is designed to control an electronic device 2 connected to the communications network 3 according to a neuronal frequency f_{ecg} received from a direct neuronal interface 4 as soon as the user U of the direct neuronal interface 4 performs an action relating to a reproduction of a visual signal sv(fn) with a given frequency fn by the connected neuronal stimulator interface 1, 1n. The action carried out by the user U is notably to look at the visual stimulus v(fn) resulting from the reproduction of a visual signal sv(fn) with the given frequency fn.

In particular, the controller 51 comprises an analyzer 510 designed to determine a given command cmdₙ of the electronic device 2 as a function of a neuronal frequency f_{ecg} received from a direct neuronal interface 4 when a visual signal sv(fn) with the given frequency fn associated with the given command cmdₙ is reproduced by the connected neuronal stimulator interface 1, 1n.

In particular, the analyzer 510 requests from the storage device 501 the given command cmdₙ associated with a given frequency fn corresponding to the received neuronal frequency f_{ecg} and supplies the given command cmdₙ intended for the electronic device 2.

In particular, the controller 51 comprises an emitter 511 of commands intended for the electronic device 2, the command emitter sending out a given command cmdₙ associated with a given frequency fn corresponding to the received neuronal frequency f_{ecg}.

In particular, the analyzer 510 supplies the given command cmdₙ to the command generator 511.

In particular, the connected-object manager 5 transmits the given command cmdₙ to the electronic device over a MQTT (“Message Queuing Telemetry Transport”) bus notably using a Wifi protocol, in particular when the connected-object manager 5 is remote from the electronic device 2.

Potentially, the controller 51 comprises a trigger 511 designed to initiate the implementation and/or the execution, by the electronic device 2, of a given command cmdₙ associated with a given frequency fn corresponding to the received neuronal frequency f_{ecg}: cmdₙ (f_{ecg}=fn).

In a first architecture (not shown), a first connected-object manager 5′ comprises the coupler 50′ and a second object manager of 5″ comprises the controller 51″.

Alternatively, in a second architecture illustrated in FIG. 2, a connected-object manager 5 comprises the coupler 50 and the controller 51. In particular, the connected-object manager 5 comprises:
the coupler 50 of a given frequency fn of a visual signal sv(fn) reproduced by the connected neuronal stimulator interface 1, 1n to a given command cmdₙ of an electronic device 2, and
the controller 51 of connected devices according to a neuronal frequency f_{ecg} received from a direct neuronal interface 4 when the visual signal sv(fn) is reproduced at the given frequency fn by the connected neuronal stimulator interface 1, 1n.

In particular, the connected-object manager 5 comprises a transmitter 54 exchanging a signal i(fn), sv(fn) relating to the given frequency fn with the connected neuronal stimulator interface 1, 1n.

In particular, the signal relating to the given frequency fn is a signal from amongst the following signals:
a signal i(fn) indicating the given frequency fn sent out by the connected-object manager 5 to the connected neuronal stimulator interface 1, 1n;
a visual signal sv(fn) to be reproduced with the given frequency fn generated by the connected-object manager 5 and sent out by the connected-object manager 5 to the connected neuronal stimulator interface 1, 1n;
a signal i(fn) indicating the given frequency fn sent out by the connected neuronal stimulator interface 1, 1n to the connected-object manager 5.

In particular, the transmitter 54 comprises an emitter 540 of the signal relating to the given frequency fn intended for the connected neuronal stimulator interface 1, 1n.

In particular, the transmitter 54 comprises a receiver 540 of the signal indicating the given frequency i(fn) coming from the connected neuronal stimulator interface 1, 1n.

In particular, the connected-object manager 5 comprises a generator 53 of a visual signal sv(fn) with a given frequency fn. The visual signal sv(fn) thus generated is designed to be reproduced by a connected neuronal stimulator interface 1, 1n. Notably, the generator 53 supplies the visual signal sv(fn) generated potentially with an identifier of the destination connected neuronal stimulator interface idₙ to the transmitter 54, in particular to the emitter 540.

A neuronal stimulator interface 1, 1n connected to a communications network 3 is designed to reproduce a visual signal sv(fn) with a given frequency fn. Potentially, the given frequency fn is associated by the connected-object manager 5 with a given command cmdₙ of an electronic device 2 connected to the communications network 3. The connected neuronal stimulator interface 1, 1n is distinct from the electronic device 2.

Thus, an electronic device 2 which is not equipped with a screen or other types of visual reproduction device may have a function referred to as BCI, in other words a neuronal interface function, added to it by means of the connected neuronal stimulator interface and of the connected-object manager according to the invention.

In particular, the connected neuronal stimulator interface 1, 1n comprises a visual reproduction device 10 designed to reproduce the visual signal sv(fn) with the given frequency fn. The visual reproduction device 10 is notably a screen (2D, 3D, etc.), one or more light-emitting or laser diodes (notably a mosaic of light-emitting diodes), a holographic display, etc.

The connected neuronal stimulator interface 1, 1n, in particular the reproduction device 10, displays a visual stimulus v(fn) with the given frequency corresponding to the reproduction of the visual signal sv(fn) with the given frequency. The visual stimulus v(fn) can be detected, seen by a user U.

The connected neuronal stimulator interface 1, 1n notably comprises a receiver 14 designed to receive:
either the signal i(fn) indicating the given frequency fn sent out by the connected-object manager 5 to the connected neuronal stimulator interface 1, 1n;
or the visual signal sv(fn) to be reproduced with the given frequency fn generated by the connected-object manager 5 and sent out by the connected-object manager 5 to the connected neuronal stimulator interface 1, 1n.

In particular, the connected neuronal stimulator interface comprises a generator 13 of a visual signal sv(fn) with a given frequency fn, for example as a function of the given frequency fn received with the signal i(fn) indicating the given frequency fn coming from the object manager 5.

The visual signal sv(fn) thus generated is designed to be reproduced by the connected neuronal stimulator interface 1, 1n. Notably, the generator 13 potentially supplies the generated visual signal sv(fn) to the reproduction device 10.

In particular, the connected neuronal stimulator interface 1, 1n and the electronic device 2 are two devices that are distinct from one another in that the connected neuronal stimulator interface 1, 1n and the electronic device 2 are separated. A connected neuronal stimulator interface 1, 1n distinct from the electronic device 2 is notably understood to mean that the electronic device 2 does not comprise, nor does it implement, the connected neuronal stimulator interface 1, 1n.

In particular, the given command cmdₙ is identical to a given command triggered by a physical button 21n of the electronic device 2. The connected neuronal stimulator interface 1, 1n is physically associated ph_ass with the physical button 21n.

Physical association is understood to mean that the connected neuronal stimulator interface is geographically placed in proximity to the physical button or to what is indicative of the physical button with which it is associated. Geographical proximity is understood to mean that the connected neuronal stimulator interface is placed directly on the physical button, or at a distance from the associated physical button that is less than the distance to the physical button of the electronic device nearest to the associated physical button, or again, if the electronic device is equipped with several devices placed on the same straight line, on a straight line perpendicular to this straight line passing through the associated physical button, etc.

Thus, the BCI interaction errors are reduced since the connected neuronal stimulator interface which reproduces the visual stimulus for the direct neuronal interaction is physically associated with the physical button of the interaction to be triggered. Hence, the user looking at the button with which he/she wishes to interact therefore sees, at the same time, the visual signal sv(fn) reproduced by the physically associated connected neuronal stimulator interface, which triggers an EEG signal with a frequency corresponding to the given frequency fn of the visual signal sv(fn) reproduced by the connected neuronal stimulator interface, itself associated with the given command of the physical button.

A physical button 21n is notably a button such as defined for FIG. 1.

In particular, in the case of an electronic device 2 equipped with several physical buttons {21i}_{i=1 . . . n . . . N} including the physical button 21n, distinct connected neuronal stimulator interfaces are associated with distinct physical buttons of the electronic device 2, including the connected neuronal stimulator interface 1n associated with the physical button 21n. Potentially, a part or even all of the physical buttons of the electronic device 2 are respectively associated with a distinct connected neuronal stimulator interface, reproducing a visual signal with a distinct given frequency.

In particular, the connected neuronal stimulator interface 1, In comprises a fastening 11 designed to keep the connected neuronal stimulator interface 1, 1n in relation to the physical button 21n.

Thus, the connected neuronal stimulator interface cannot move, limiting the interaction errors linked to the loss of the physical association of the connected neuronal stimulator interface with the physical button the given command of which is associated with the given frequency fn of the visual signal sv(fn) reproduced by the connected neuronal stimulator interface.

Advantageously, the connected neuronal stimulator interface 1, 1n comprises a rechargeable battery 12. Notably, the connected neuronal stimulator interface 1, In may be connected as a power supply to any given electronic device such as the electronic device 2, a computer, a tablet, etc. notably by means of a USB cable in order to be recharged, or an electrical socket via a battery charger.

An electronic device 2 connected to a communications network 3 comprises an actuator 20n designed to control a component of the electronic device 2 as a function of at least one given command cmdₙ, for example supplied by a connected-object manager 5. The given command cmdₙ depends on a neuronal frequency f_{ecg} emitted by a direct neuronal interface 4 when a visual signal sv(fn) is reproduced with a given frequency fn by a connected neuronal stimulator interface 1, 1n. The neuronal frequency f_{ecg} emitted is a function of the given frequency fn.

Notably, the actuator 20n is a processor 20n designed to execute a processing operation as a function of at least one command.

In particular, the electronic device 2 comprises a control interface 24 designed to receive a given command from a connected-object manager 5 via the communications network 3 as a function of a neuronal frequency f_{ecg} received from a direct neuronal interface 4 when a visual signal sv(fn) is reproduced with a given frequency fn by the connected neuronal stimulator interface 1. The neuronal frequency f_{ecg} received is a function of the given frequency fn.

In particular, the electronic device 2 comprises a power supply 22 of the connected neuronal stimulator interface when the connected neuronal stimulator interface 1, 1n is associated with a physical button 21n of the electronic device 2.

In particular, the power supply 22 implemented in the electronic device 2 recharges the battery 12 of the connected neuronal stimulator interface 1, 1n notably when the connected neuronal stimulator interface 1, 1n is connected to the electronic device 2, for example, by a cable, by induction, etc.

In particular, the power supply 22 of the connected neuronal stimulator interface is a power supply from amongst the following power supplies:
a power supply connector designed to be connected to the connected neuronal stimulator interface when the connected neuronal stimulator interface is associated with a physical button of the electronic device;
a charger by induction designed to recharge a battery of the connected neuronal stimulator interface when the connected neuronal stimulator interface is associated with a physical button of the electronic device.

A direct neuronal interface 4 is designed to capture a frequency f_{ecg} relating to a user U when a visual signal sv(fn) is reproduced with a given frequency fn. The direct neuronal interface 4 comprises an emitter 44 (not shown) of the captured neuronal frequency f_{ecg} to a connected-object manager via a communications network 3. The emitter of the captured neuronal frequency is designed to trigger a given command cmdₙ of an electronic device 2 associated with the given frequency fn of a visual signal sv(fn) reproduced by a neuronal stimulator interface 1, 1n connected to the communications network 3. The neuronal frequency f_{ecg} emitted is a function of the given frequency fn.

In particular, the direct neuronal interface 4 is able to be connected to a connected-object manager 5.

In particular, a direct neuronal interface 4 is designed to supply a captured neuronal frequency f_{ecg} when a visual signal sv(fn) is reproduced with a given frequency fn. The direct neuronal interface 4 comprises a communication interface 44 designed to be connected to a connected-object manager 5. The communication interface 44 is designed to transmit to the connected-object manager 5 the captured neuronal frequency f_{ecg} when a visual signal sv(fn) is reproduced with a given frequency fn by the connected neuronal stimulator interface 1.

In particular, the emitter 44 is a communication interface with the communications network 3 connected to the connected-object manager 5.

In particular, since the given frequency fn is associated by the connected-object manager 5 with a given command cmdₙ of the electronic device 2, the captured neuronal frequency f_{ecg} corresponding to the given frequency fn of the reproduced visual signal being observed by the user U is therefore associated with this same command cmdₙ of the electronic device 2. The captured neuronal frequency is accordingly able to control cmdₙ the electronic device.

In particular, the emitter 44 is designed to supply the captured neuronal frequency f_{ecg} relating to a user U when a visual signal sv(fn) is reproduced with a given frequency fn by a neuronal stimulator interface 1, 1n connected to a communications network 3.

The captured neuronal frequency f_{ecg} is a frequency of a visually evoked signal potential within signals of the electroencephalogram of a user U.

A given frequency fn of reproduction of a visual signal by a neuronal stimulator interface 1 connected to a communications network 3 is associated, by the connected-object manager 5, with a given command cmdₙ of an electronic device 2 connected to the communications network 3. The connected neuronal stimulator interface 1 and the electronic device are distinct from one another.

Potentially, the connected neuronal stimulator interface 1, 1n is physically associated ph_ass with a physical button 21n of the electronic device, where operation of the physical button triggers the given command cmdₙ.

In particular, in parallel with the physical association ph_ass, the connected-object manager 5 associates, notably by means of a coupler 50, the given command cmdₙ corresponding to the physical button 21n with a given frequency fn usable by the connected neuronal stimulator interface 1 for reproducing a visual signal sv(fn).

In particular, the connected-object manager 5 transmits, notably by means of a transmitter 54 and/or, more particularly of an emitter 540, to the connected neuronal stimulator interface 1, 1n a visual signal sv(fn) with the given frequency or a signal i(fn) indicating the given frequency. The visual signal sv(fn) with the given frequency transmitted by the connected-object manager 5 is potentially generated by the connected-object manager 5, notably by a visual signal generator 53.

In particular, the reproduction device 10 of the connected neuronal stimulator interface 1, 1n reproduces the visual signal sv(fn) with the given frequency, notably after receiving the visual signal sv(fn) with the given frequency or a signal i(fn) indicating the given frequency, notably by means of a receiver 14. Potentially, the connected neuronal stimulator interface 1 reproduces, in particular by a visual signal generator 13, the visual signal sv(fn) with the given frequency according to the signal i(fn) indicating given frequency received from the connected-object manager.

When the user U looks at the visual stimulus v(fn) resulting from the reproduction of the visual signal sv(fn) with the given frequency fn by the connected neuronal stimulator interface 1, 1n, a direct neuronal interface 4 captures a neuronal frequency f_{ecg} relating to the user U. The captured neuronal frequency f_{ecg} and the given frequency fn have the same value.

In particular, the direct neuronal interface 4 is implemented in a BCI headset or an augmented reality headset.

The direct neuronal interface 4 supplies, notably by means of an emitter 44, the captured neuronal frequency f_{ecg} via the communications network 3, in particular to the connected-object manager 5. A given command cmdₙ being a function of the captured neuronal frequency is implemented by the electronic device 2.

Potentially, the electronic device 2 receives from a controller 51, in particular from an analyzer 510, notably implemented in the connected-object manager 5, the given command cmdₙ depending on the neuronal frequency sent out by the direct neuronal interface 4 and on the pair associating a given command with a given frequency (cmdₙ,fn).

In particular, a communications terminal of the user comprises a configuration interface (not shown) of the connected-object manager 5. This configuration interface allows the user to control the coupler 50 of the connected-object manager to associate a given command cmdₙ of the electronic device 2 with a given frequency fn of the connected neuronal stimulator interface 1. The configuration interface is notably implemented on the communications terminal in the form of an application.

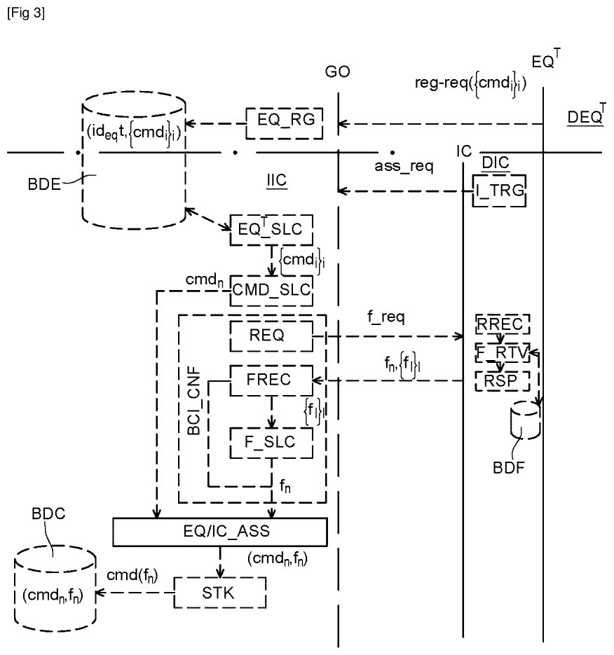

FIG. 3 illustrates a simplified diagram of exchanges during the implementation of a method for initializing a connected neuronal stimulator interface according to the invention.

A method IIC for initializing a neuronal stimulator interface IC connected to a communications network and an electronic device EQt connected to the communications network comprises a pairing EQ/IC_ASS associating a given frequency fn of a visual signal sv(fn) reproduced by a neuronal stimulator interface IC connected to the communications network with a given command cmdₙ of an electronic device EQt connected to the communications network.

In particular, the initialization method IIC comprises, prior to the pairing EQ/IC_ASS, a selection EQt_SLC of a connected electronic device notably from amongst several connected devices notably managed by a connected-object manager GO. The selection of electronic device EQt_SLC notably provides the commands {cmdᵢ}i designed to be implemented by the selected electronic device EQt. Potentially, the selection of electronic device EQt_SLC obtains the list of the commands {cmdᵢ}, able to be implemented by the selected electronic device EQt either from the selected electronic device EQt (option not shown), or from a storage device, notably a database of electronic devices BDE potentially implemented in the connected-object manager GO.

In particular, the initialization method IIC comprises, prior to the pairing EQ/IC_ASS, a selection of a command of an electronic device CMD_SLC, in particular within a list of commands {cmdᵢ}, of a previously selected electronic device EQt_SLC. In the case where a connected neuronal stimulator interface IC is physically associated with a physical button of the electronic device EQt, the selected command cmdₙ, also referred to as given command, corresponds to the command triggered by an action of a user U on the physical button of the electronic device EQt.

In particular, the initialization method IIC comprises, prior to the pairing EQ/IC_ASS, a neuronal configuration BCI_CNF supplying a given frequency fn able to be used by the connected neuronal stimulator interface IC in the process of association with the electronic device EQt.

In particular, the initialization method IIC comprises, prior to the pairing EQ/IC_ASS, a frequency receipt FREC receiving from the connected neuronal stimulator interface IC: either a list of frequencies {fl]ₗ, able to be used by the connected neuronal stimulator interface IC for reproducing a visual signal, or the given frequency fn. In particular, the neuronal configuration BCI_CNF comprises the frequency receipt FREC.

In particular, the initialization method IIC comprises, prior to the frequency receipt FREC, a frequency request REQ sending out, and potentially generating prior to sending out, a frequency request f_req to the connected neuronal stimulator interface IC. In particular, the neuronal configuration BCI_CNF comprises the frequency request REQ prior to the frequency receipt FREC.

In particular, the initialization method IIC comprises, prior to the pairing EQ/IC_ASS, a selection F_SLC of a given frequency fn from amongst the frequencies {fl]ₗ able to be used by the connected neuronal stimulator interface IC in the process of association with the electronic device EQt. In particular, the neuronal configuration BCI_CNF comprises the given frequency selection F_SLC.

In particular, the initialization method IIC comprises a storage STCK of the given command-given frequency pair (cmdₙ,fn) supplied by the pairing EQ/IC_ASS. The storage STCK records the given command-given frequency pair (cmdₙ,fn) in a storage device, notably a database of neuronal commands BDC.

In particular, the initialization method IIC is triggered by a request for association ass_req of the connected neuronal stimulator interface IC with the electronic device EQt. Notably, the connected neuronal stimulator interface IC implements an initialization trigger I_TRG sending out the association request ass_req.

In particular, a method for declaration of a connected neuronal stimulator interface DIC is implemented by the connected neuronal stimulator interface IC. The method for declaration of a connected neuronal stimulator interface DIC comprises the initialization trigger I_TRG.

Potentially, the method for declaration of a connected neuronal stimulator interface DIC comprises a frequency declaration RSP sending to the initialization method, notably upon frequency receipt FREC, either a list of frequencies {fl]ₗ, able to be used by the connected neuronal stimulator interface IC for reproducing a visual signal, or the given frequency fn.

In particular, the method for declaration of a connected neuronal stimulator interface DIC comprises, prior to the frequency declaration RSP, a search F_RTV for the frequencies usable by the connected neuronal stimulator interface IC. The frequency search F_RTV reads the usable frequencies previously recorded in a storage device, notably a database of frequencies BDF potentially implemented in the connected neuronal stimulator interface IC.

In particular, the method for declaration of a connected neuronal stimulator interface DIC comprises, prior to the frequency declaration RSP and, where relevant, prior to the frequency search F_RTV, a receiver of frequency requests RREC coming from the initialization method IIC.

In one particular embodiment, prior to the initialization method IIC, an electronic device EQt implements a device declaration method DEQt. The device declaration method DEQt sends, via the communications network to which it is connected, a registration request reg_req to a device registration method EQ_REG notably implemented by the connected-object manager GO. The registration request notably comprises a list of the commands {cmdᵢ}i able to be implemented by the electronic device EQt.: reg_req({cmdᵢ}i).

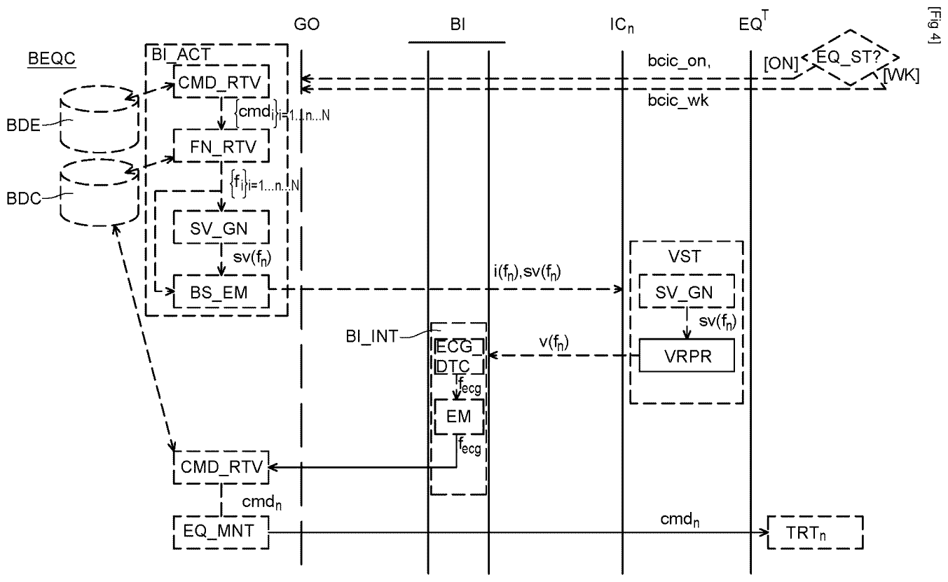

FIG. 4 illustrates a simplified diagram of exchanges during the implementation of a method for controlling an electronic device using a connected neuronal stimulator interface according to the invention.

A method BEQC for controlling an electronic device controls cmdₙ an electronic device EQt connected to a communications network according to a neuronal frequency f_{ecg} received from a direct neuronal interface BI when a visual signal sv(fn) is reproduced with a given frequency fn by a neuronal stimulator interface IC connected to the communications network. The received neuronal frequency f_{ecg} is a function of the given frequency fn, the given frequency and the given command being associated during an initialization of the neuronal stimulator interface.

In particular, the control method BEQC is implemented by a connected-object manager GO.

In particular, the control method BEQC comprises an activation BI_ACT of a reproduction VRPR of a visual signal sv(fn) with a given frequency fn notably implemented by a connected neuronal stimulator interface ICn. A visual stimulation method VST, notably implemented by the connected neuronal stimulator interface ICn, comprises the reproduction VRPR of the visual signal sv(fn) with a given frequency fn.

In particular, the activation BI_ACT of a reproduction of a visual signal sv(fn) with a given frequency comprises a neuronal stimulation emitter BS_EM emitting a signal.

In particular, the triggering BS_EM emits a signal relating to the given frequency fn used during the reproduction VRPR of a visual signal with the given frequency fn. The signal emitted by the triggering BS_EM is notably:
either a signal i(fn) indicating the given frequency fn intended for the reproduction VRPR and/or for the visual stimulation method VST;
or a visual signal sv(fn) to be reproduced with the given frequency fn of the reproduction VRPR and/or of the visual stimulation method VST.

In particular, the visual stimulation method VST comprises, prior to the reproduction VRPR, a generation SV_GN of visual signal sv(fn) with a given frequency fn indicated in the indication signal i(fn) coming from the triggering BS_EM.

In particular, the method for controlling the electronic device BEQC comprises, prior to the triggering BS_EM of reproduction of a visual signal sv(fn) with a given frequency, a search FN_RTV of the given frequencies associated with the commands of the electronic device EQt, in particular, the search of given frequencies FN_RTV, notably in a storage device such as a database of commands BDC, for example the database of commands in FIG. 3.

In particular, the method for controlling the electronic device BEQC comprises, prior to the triggering BS_EM of reproduction of a visual signal sv(fn) with a given frequency, a generation SV_GN of a visual signal sv(fn) with a given frequency fn indicated in the indication signal i(fn) coming from the triggering BS_EM.

In particular, the method for controlling an electronic device BECQ comprises, prior to the search for a given frequency FN_RTV, a search of the commands of the electronic device CMD_RTV, notably in a storage device such as a database of electronic devices BDE, for example the database of electronic devices in FIG. 3, supplying a list of commands {cmdᵢ}_{i=1 . . . n . . . N} able to be implemented by the electronic device EQt.

Potentially, the search of commands CMD_RTV is triggered by an activation command bcic_on or a wake-up command bcic_wk for one or more connected neuronal stimulator interfaces ICn associated with the electronic device EQt.

For example, a verification of status of the electronic device EQ_ST? determines whether the device goes from an “OFF” or “deactivated” status to an “ON” or “activated” status.

When a switch from the “OFF” or “deactivated” status to an “ON” or “activated” status is detected [ON] by this verification EQ_ST?, then the verification EQ_ST? generates an activation command bcic_on. The activation command bcic_on potentially comprises the list of commands supplying a list of commands {cmdᵢ}_{i=1 . . . n . . . N} able to be implemented by the electronic device EQt. The action command bcic_on triggers the activation BI_ACT of a reproduction VRPR of a visual signal sv(fn) with a given frequency fn and/or the search of commands CMD_RTV.

When a switch from the “standby” status to an “ON” or “activated” status is detected [WK] by this verification EQ_ST?, then the verification EQ_ST? generates a wake-up command bcic_wk. The wake-up command bcic_wk potentially comprises the list of commands supplying a list of commands {cmdᵢ}_{i=1 . . . n . . . N} able to be implemented by the electronic device EQt. The wake-up command bcic_wk triggers the activation BI_ACT of a reproduction VRPR of a visual signal sv(fn) with a given frequency fn and/or the search of commands CMD_RTV.

In particular, the method for controlling an electronic device BEQC comprises, after triggering BS_EM a reproduction of a visual signal sv(fn) with a given frequency, a supervision of the electronic device EQ_MNT controlling the electronic device EQt by means of a given command cmdₙ being dependent on a captured neuronal frequency f_{ecg} coming from a direct neuronal interface BI during the reproduction VRPR of the visual signal sv(fn) with a given frequency by a connected neuronal stimulator interface ICn associated with the electronic device EQt.

In particular, the method for controlling an electronic device BEQC comprises, after triggering BS_EM a reproduction of a visual signal sv(fn) with a given frequency and prior to the given command cmdₙ of the electronic device EQt, notably the generation of the given command cmdₙ by the supervision of the electronic device EQ_MNT, a search of given commands CMDN_RTV as a function of a captured neuronal frequency f_{ecg} coming from a direct neuronal interface BI during the reproduction VRPR of the visual signal sv(fn) with a given frequency by a connected neuronal stimulator interface ICn associated with the electronic device EQt. The search for a given command CMDN_RTV is carried out in a storage device such as the database of commands BDC, for example the database of commands in FIG. 3.

In particular, a neuronal interaction method BI_INT provides the captured neuronal frequency f_{ecg} coming from a direct neuronal interface BI during the reproduction VRPR of the visual signal sv(fn) with a given frequency by a connected neuronal stimulator interface ICn associated with the electronic device EQt.

The neuronal interaction method BI_INT is notably implemented by the direct neuronal interface BI.

In particular, a neuronal frequency detection ECG_DTC supplies, during the reproduction VRPR of the visual signal sv(fn) with a given frequency by a connected neuronal stimulator interface ICn associated with the electronic device EQt, the captured neuronal frequency. The neuronal frequency detection is notably activated by an action of the user U of the direct neuronal interface BI such as the fact that the user U is looking at the visual stimulus v(fn) resulting from the reproduction VRPR of a visual signal sv(fn) with the given frequency.

In particular, the neuronal interaction method BI_INT comprises the neuronal frequency detection ECG_DTC.

In particular, an emission of a captured neuronal frequency EM transmits to the method for controlling electronic devices BEQC the neuronal frequency f_{ecg} captured during the reproduction VRPR of the visual signal sv(fn) with a given frequency by a connected neuronal stimulator interface ICn associated with the electronic device EQt. Notably, the captured neuronal frequency f_{ecg} emitted by the emission EM is provided by the neuronal frequency detection ECG_DTC.

In particular, the neuronal interaction method BI_INT comprises the emission of the captured neuronal frequency EM.

In particular, the given command cmdₙ triggers the execution of a given processing operation TRTn by the electronic device EQt.

In one particular embodiment, a connected-object management method OCMNGT comprises a method IIC for initializing a connected neuronal stimulator interface and a method for controlling electronic devices BEQC. For a specific electronic device, the management method OCMNGT implements the method IIC for initializing at least one connected neuronal stimulator interface associated with this specific electronic device prior to the implementation of the control method BEQC for this specific electronic device.

In one embodiment of the method IIC for initializing a connected neuronal stimulator interface and/or the method BEQC for controlling an electronic device and/or the connected-object management method OCMNGT, there is a program comprising program code instructions for the execution of the steps of the method for initializing a connected neuronal stimulator interface and/or the method for controlling an electronic device and/or the connected-object management method when said program is executed by a processor.

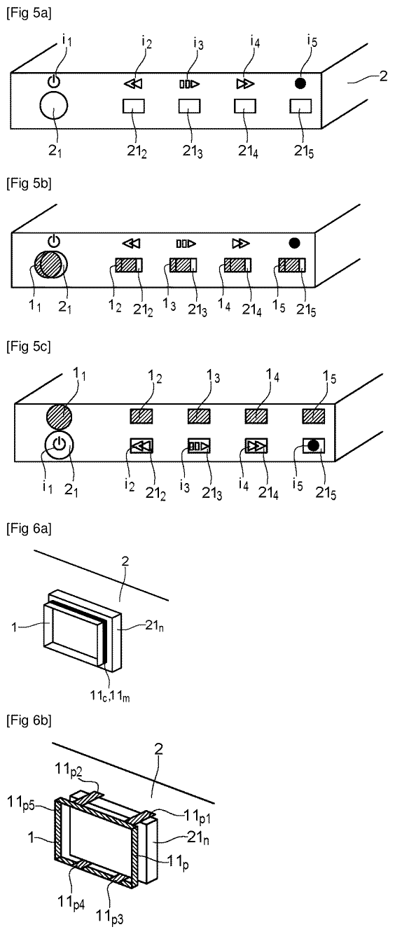

FIGS. 5a to 5c illustrate an electronic device and various physical associations of connected neuronal stimulator interfaces with this electronic device.

FIG. 5a illustrates a simplified diagram of an electronic device for which the invention may be used.

The electronic device 2 comprises several physical buttons 21₁, 21₂, 21₃, 21₄, 21₅ potentially with associated indicators i₁, i₂, i₃, i₄, i₅. The electronic device is notably a DVD player comprising:
a physical button 21₁ for start/stop or for standby/wake-up as indicated by the associated indicator i₁;
a physical button 21₂ for fast rewind as indicated by the associated indicator i₂;
a physical button 21₃ for read/pause as indicated by the associated indicator i₃;
a physical button 21₄ for fast forward as indicated by the associated indicator i₄;
a physical button 21₅ for record as indicated by the associated indicator is (red LED).

In another exemplary embodiment (not shown), the electronic device 2 is a TV box or TV decoder (TNT, Internet, etc.) allowing the access by a television to TV channels via the TNT, Internet, etc. to be activated and channels to be changed via physical buttons present on the TV box. The TV box would have at least three physical buttons:
a physical button for start/stop or for standby/wake-up also referred to as “ON/OFF” button,
a physical button associated with a “+” sign for going to the next channel in a list of the channels offered by the TV box;
a physical button associated with a “−” sign for going to the previous channel in a list of the channels offered by the TV box.

FIG. 5b illustrates a simplified diagram of a first physical association of connected neuronal stimulator interfaces with an electronic device according to the invention.

In the example in this FIG. 5b, each of the physical buttons 21₁ . . . 21₅ is associated with a connected neuronal stimulator interface 1₁ . . . 1₅: (21₁, 1₁), (21₂, 1₂), (21₃, 1₃), (21₄, 1₄), (21₅, 1₅). In addition, the physical association is linked to the positioning of the connected neuronal stimulator interface 1₁ . . . 1₅ directly on the physical button 21₁ . . . 21₅ with which it is associated.

This physical association has been chosen in this example because the indicator i₁ . . . i₅ associated with the physical button 21₁ . . . 21₅ is placed, rather than on the physical button itself, on the electronic device 2 above the associated physical button.

The connected neuronal stimulator interface covers notably all or part of the physical button with which it is physically associated.

FIG. 5c illustrates a simplified diagram of a second physical association of connected neuronal stimulator interfaces with an electronic device according to the invention.

In the example of this FIG. 5c, each of the physical buttons 21₁ . . . 21₅ is associated with a connected neuronal stimulator interface 1₁ . . . 1₅: (21₁, 1₁), (21₂, 1₂), (21₃, 1₃), (21₄, 1₄), (21₅, 1₅). In addition, the physical association is linked to the positioning of the connected neuronal stimulator interface 11 . . . 15 on the electronic device 2 next to the physical button 21₁ . . . 21₅ with which it is associated, notably above the associated physical button.

This physical association has been chosen in this example because the indicator i₁ . . . 1₅ associated with the physical button 21₁ . . . 21₅ is placed directly on the physical button itself.

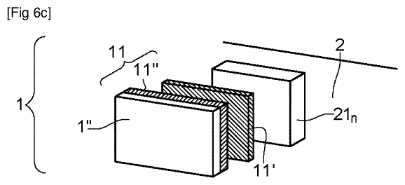

In order to give the DVD player and/or the TV box in the above examples neuronal interaction or BCI functionalities, at least one of the buttons of the DVD player and/or of the TV box is associated with a connected neuronal stimulator interface, notably in the form of a bondable connected neuronal stimulator interface, also called BCI stickers, as is illustrated in FIG. 6a. The BCI sticker(s) is (are) added:
either directly onto the button(s) with which the user wishes to make the command actionable by means of a direct neuronal interface as illustrated in FIG. 5b for the DVD player,
or next to the button(s) with which the user wishes to make the command actionable by means of a direct neuronal interface as illustrated in FIG. 5c for the DVD player,
or at another location, in particular associated with the indicator of the button with which the user wishes to make the command actionable by means of a direct neuronal interface (not shown). In these examples linked to the television (DVD player, TV box), a metal or magnetic ruler may be positioned next to the television onto which an indicator is affixed corresponding to the button with which the user wishes to make the command actionable by means of a direct neuronal interface and the BCI sticker associated with this command.

Notably, in the example of the DVD player, one or more of the following BCI stickers are applied to the DVD player:
a first sticker 1₁ is associated with the command triggerable by means of the on/off or standby/wake-up physical button 21₁;
a second sticker 1₂ associated with the command triggerable by means of the fast rewind physical button 21₂;
a third sticker 1₃ associated with the command triggerable by means of the read/pause physical button 21₃;
a fourth sticker 1₄ associated with the command triggerable by means of the fast forward physical button 21₄;
a fifth sticker 1₅ associated with the command triggerable by means of the record physical button 21₅.

Potentially, in the example of the TV box or TV decoder (TNT, Internet, etc.), one or more of the following BCI stickers are applied to the TV box:
a first sticker is associated with the command triggerable by means of the start/stop or standby/wake-up physical button also called “ON/OFF” button,
a second sticker is associated with the command triggerable by means of the “+” physical button for switching to the next channel in a list of the channels offered by the TV box;
a third sticker is associated with the command triggerable by means of the “−” physical button for switching to the previous channel in a list of the channels offered by the TV box.

Thus, the association between the command and the connected neuronal stimulator interface is intuitive and automatic.

FIGS. 6a to 6c illustrate various attachment systems for the connected neuronal stimulator interface according to the invention.

In particular, the connected neuronal stimulator interface 1 comprises a fastening 11 designed to keep the connected neuronal stimulator interface 1 in relation to the physical button 21n.

FIG. 6a illustrates a simplified diagram of a first variant of a connected neuronal stimulator interface comprising a system for fixing by bonding according to the invention.

An electronic device 2 comprises at least one physical button 21n.

The connected neuronal stimulator interface 1 comprises a fastening 11c, 11m allowing the connected neuronal stimulator interface 1 to be fixed onto the electronic device 2 above or directly on (as illustrated in FIG. 6a) a physical button 21n the given command cmdₙ of which is associated with the given frequency fn of reproduction of a visual signal sv(fn) by the connected neuronal stimulator interface 1.

The fastening is notably a fastening by adhesive 11c, such as a double-sided tape, or by magnetism 11m. For example, when the electronic device 2 or the physical button 21n is metal or covered by a metal sticker, the fastening 11m comprises a magnet for attaching the connected neuronal stimulator interface 1 respectively to the electronic device next to the physical button or directly onto the physical button. Conversely, when the electronic device 2 or the physical button 21n comprises a magnetic outer surface or is covered by a magnetic sticker, the fastening 11m is metal. The magnetic or metal attachment system 11m then allows a bonding by magnetism onto the physical button 21n as illustrated in FIG. 6a or onto the electronic device 2 next to the physical button 21n.

One exemplary embodiment would be to use connected neuronal stimulator interfaces in the form of stickers. The stickers would be stuck onto the physical buttons that the user wishes to control by neuronal interface or BCI.

FIG. 6b illustrates a simplified diagram of a second variant of a connected neuronal stimulator interface comprising a clip-on attachment system according to the invention.

An electronic device 2 comprises at least one physical button 21n.

The connected neuronal stimulator interface 1 comprises a fastening 11p allowing the connected neuronal stimulator interface 1 to be fixed onto the electronic device 2 above or directly onto (as illustrated in FIG. 6b) a physical button 21n the given command cmdₙ of which is associated with the given frequency fn of reproduction of a visual signal sv(fn) by the connected neuronal stimulator interface 1.

The fastening is notably a clip-on attachment system 11p such as a sucker, fins or spigots 11p1, 11p2, 11p3, 11p4 (see FIG. 6b), etc., allowing the connected neuronal stimulator interface 1 to be slid onto the associated physical button 21n itself or next to this physical button onto the electronic device 2.

FIG. 6c illustrates a simplified diagram of a third variant of a connected neuronal stimulator interface comprising a first part designed to reproduce a visual signal sv(fn) with a given frequency fn and a second part comprising a fastening according to the invention.

An electronic device 2 comprises at least one physical button 21n.

The connected neuronal stimulator interface 1 comprises a fastening 11 allowing the connected neuronal stimulator interface 1 to be attached to the electronic device 2 above or directly on (as illustrated in FIG. 6b) a physical button 21n the given command cmdₙ of which is associated with the given frequency fn of reproduction of a visual signal sv(fn) by the connected neuronal stimulator interface 1.

In particular, the fastening 11 is composed of two separable parts:
a first part 11′ called support designed to keep the connected neuronal stimulator interface 1 in relation to the physical button 21n and
a second part 11″ fixable onto the support 11′, being the interface 1″ proper designed to reproduce a visual signal sv(fn) with a given frequency fn comprising the second part 11″ of the fastening 11.

The second part 11″ may be attached in a removable manner to the first part 11′ of the fastening 11.

Thus, the connected neuronal stimulator interface may be moved notably in order to be recharged and repositioned exactly in physical association with the physical button with which it was previously associated.

The invention is also aimed at a medium. The information medium may be any given entity or device capable of storing the program or even the programs. For example, the medium may comprise a storage means, such as a ROM, for example a CD ROM or a microelectronic circuit ROM or else a magnetic recording means, for example a diskette or a hard disk.

On the other hand, the information medium may be a transmissible medium such as an electrical or optical signal which may be carried via an electrical or optical cable, by radio or by other means. The program according to the invention may, in particular, be up/downloaded over a network notably of the Internet type.

Alternatively, the information medium may be an integrated circuit into which the program is incorporated, the circuit being designed to execute or to be used in the execution of the method in question.

In another embodiment, the invention is implemented by means of software and/or hardware components. In this respect, the term module may just as easily correspond to a software component or to a hardware component. A software component corresponds to one or more computer programs, one or more sub-programs of a program or, more generally, to any element of a program or of a software application designed to implement a function or a set of functions according to the description hereinabove. A hardware component corresponds to any element of a hardware assembly designed to implement a function or a set of functions.

1. A neuronal stimulator interface comprising:
a receiver configured to connect to a communications network; and
a visual reproduction device designed to reproduce a visual signal with a given frequency, the given frequency being associated with a given command of an electronic device connected to the communications network during an initialization of the neuronal stimulator interface, in which the neuronal stimulator interface is distinct from the electronic device.

2. The connected neuronal stimulator interface as claimed in claim 1, in which the given command is identical to a given command triggered by a physical button of the electronic device, the connected neuronal stimulator interface being physically associated with the physical button.

3. The connected neuronal stimulator interface as claimed in claim 2, in which the connected neuronal stimulator interface comprises a fastener to keep the connected neuronal stimulator interface in a physical position in relation to the physical button.

4. The connected neuronal stimulator interface as claimed in claim 3, in which the fastener is composed of first and second separable parts, including:
a first part referred to as support to keep the connected neuronal stimulator interface in relation to the physical button; and
a second part fixable onto the support, wherein the visual reproduction device comprises the second part of the fastener.

5. The connected neuronal stimulator interface as claimed in claim 1, in which the connected neuronal stimulator interface comprises a rechargeable battery.

6. A connected-object managing device, comprising:
a transmitter configured to connect to objects that are connected to a communications network including at least one connected neuronal stimulator interface and at least one electronic device;
at least one processor; and
at least one non-transitory computer readable medium comprising instructions stored thereon which when executed by the at least one processor configure the connected-object managing device to:
connect to the at least one connected neuronal stimulator interface and the at least one electronic device through the transmitter; and
associate in a database a given frequency of a visual signal reproduced by at least one of the at least one neuronal stimulator interface connected to the communications network with a given command of at least one of the at least one electronic device connected to the communications network.

7. A connected-object managing device comprising:
at least one processor; and
at least one non-transitory computer readable medium comprising instructions stored thereon which when executed by the at least one processor configure the connected-object managing device to:
connect to objects that are connected to a communications network including at least one connected neuronal stimulator interface and at least one electronic device;
control, by using of a given command, at least one of the at least one electronic device connected to the communications network as a function of a neuronal frequency received from a direct neuronal interface in response to a visual signal with a given frequency being reproduced by at least one of the at least one connected neuronal stimulator interface, the received neuronal frequency being a function of the given frequency, the given frequency and the given command being associated.

8. (canceled)

9. The connected-object manager as claimed in claim 6, in which the transmitter is configured to exchange a signal relating to the given frequency with at least one of the at least one connected neuronal stimulator interface from amongst the following signals:
a signal indicating the given frequency sent out by the connected-object manager to the connected neuronal stimulator interface;
a visual signal to be reproduced with the given frequency generated by the connected-object manager and sent by the connected-object manager to the connected neuronal stimulator interface;
a signal indicating the given frequency sent out by the connected neuronal stimulator interface to the connected-object manager.

10. (canceled)

11. An electronic device comprising:
a control interface configured to connect to a communications network;
an actuator which controls a component of the electronic device according to at least one given command received on the control interface, the given command being a function of a neuronal frequency emitted by a direct neuronal interface in response to a visual signal with a given frequency being reproduced by a connected neuronal stimulator interface, the emitted neuronal frequency being a function of the given frequency, the given frequency and the given command being associated during an initialization of the neuronal stimulator interface.

12. The electronic device as claimed in claim 11, in which the electronic device comprises a power supply of the connected neuronal stimulator interface when the connected neuronal stimulator interface is associated with a physical button of the electronic device from amongst the following power supplies:
a power supply connector designed to be connected to the connected neuronal stimulator interface when the connected neuronal stimulator interface is associated with a physical button of the electronic device;
a charger by induction designed to recharge a battery of the connected neuronal stimulator interface when the connected neuronal stimulator interface is associated with a physical button of the electronic device.

13. A method for initializing a neuronal stimulator interface connected to a communications network and an electronic device connected to the communications network, in which the method comprises:
performing a pairing which associates a given frequency of a visual signal reproduced by the neuronal stimulator interface connected to the communications network with a given command of the electronic device connected to the communications network; and
storing the pairing in a database.

14. A control method for controlling an electronic device, in which the control method comprises:
receiving a neuronal frequency from a direct neuronal interface in response to a visual signal with a given frequency being reproduced by the neuronal stimulator interface;
transmitting a given command, to an electronic device over a communications network as a function of the received neuronal frequency, the received neuronal frequency being a function of the given frequency, the given frequency and the given command being associated during an initialization of the neuronal stimulator interface.

15. A non-transitory computer readable medium comprising a program stored thereon comprising program code instructions for execution of a method for initializing a connected neuronal stimulator interface when said program is executed by a processor, the neuronal stimulator interface being connected to a communications network, wherein the method comprises:
performing a pairing which associates a given frequency of a visual signal reproduced by the neuronal stimulator interface connected to the communications network with a given command of an electronic device connected to the communications network; and
storing the pairing in a database.

16. The connected-object manager as claimed in claim 7, wherein the connected-object managing device further comprises a transmitter configured to exchange a signal relating to the given frequency with at least one of the at least one connected neuronal stimulator interface from amongst the following signals:
a signal indicating the given frequency sent out by the connected-object manager to the connected neuronal stimulator interface;
a visual signal to be reproduced with the given frequency generated by the connected-object manager and sent by the connected-object manager to the connected neuronal stimulator interface;
a signal indicating the given frequency sent out by the connected neuronal stimulator interface to the connected-object manager.
